(19) United States
(12) Patent Application Publication
(10) Pub. No.: US 2004/0067959 A1
(43) Pub. Date: Apr. 8, 2004

(54) Morphinoid derivatives as delta-opioid agonists and antagonists

(76) Inventors: Giulio Dondio, Milan (IT); Stefania Gagliardi, Milan (IT); Davide Graziani, Milan (IT); Luca Francesco, Milan (IT)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/398,313

(22) PCT Filed: Oct. 10, 2001
(86) PCT No.: PCT/EP01/11558

(30) Foreign Application Priority Data
Oct. 12, 2000 (GB) 0025056.3

(51) Int. Cl. A61K 31/496; C07D 489/00

(52) U.S. Cl. 514/253.02; 544/361

(57) ABSTRACT

Compounds of formula (I) in which R₁ is hydrogen or alkyl; R₂ is hydrogen or one or more alkyl groups; R₃ is R₁ or R₁X—; wherein R₁ is hydrogen or optionally substituted alkyl, aryl, arylalkyl, cycloalkyl or heterocyclyl and X is a linking group; and R₄ is hydrogen or alkyl; with the proviso that when R₄ is methyl and R₃ is methyl or hydroxyethyl then R₂ is not hydrogen. Are selective delta opioid receptor ligands and therefore of potential therapeutic utility as analgesics and antihyperalgesics for different pain conditions; immunosuppressants to prevent rejection in organ transplant and skin grafts; anti-allergic and anti-inflammatory agents; brain cell protectants; agents for treating drug and alcohol abuse, gastritis, diarrhoea, cardiovascular and respiratory diseases, cough, mental illness and epilepsy.

The present invention is concerned with novel morphinoid compounds, processes for their preparation and their use in medicine.

The presence of at least three populations of opioid receptors (mu, delta and kappa) is now well established and documented and all three appear to be present in the central and peripheral nervous system of many species including man (J. A. H. Lord et al., Nature 1977, 267, 495).

Activation of all three opioid receptor subtypes can lead to antinociception in animal models. In particular, studies with peptidic delta agonists have indicated that activation of the delta receptor produces antinociception in rodents, primates and can induce clinical analgesia in man (D. E. Moulin et al. Pain, 1985, 23 213). Evidence exists that suggest a lesser propensity of delta agonists to cause the usual side-effects associated with mu and kappa activation (Galligan et al, J. Pharm. Exp. Ther., 1984, 229, 641).

WO 96/02545 and WO 97/25331 (SmithKline Beecham SpA) disclose substituted monoheterocycle-condensed morphinoid derivatives which are potent and selective delta opioid agonists and antagonists, including (in WO 97/25331) the compounds:

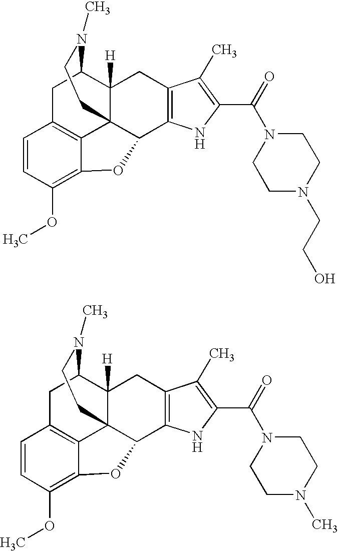

We have discovered that certain novel morphinoid compounds are surprisingly more potent and selective than known delta opioid receptor ligands and therefore of potential therapeutic utility as analgesics and antihyperalgesics for different pain condition; immunosuppressants to prevent rejection following organ transplants and skin grafts; anti-allergic and anti-inflammatory agents; brain cell protectants; agents for treating drug and alcohol abuse, cardiovascular and respiratory diseases, cough, mental illness and epilepsy; agents for treating gastrointestinal disorders such as gastritis, diarrhoea and irritable bowel syndrome; and, in general, for the treatment of those pathological conditions which customarily can be treated with agonists and antagonists of the delta opioid receptor.

According to the present invention, there is provided a compound, or a solvate or salt thereof, of formula (I):

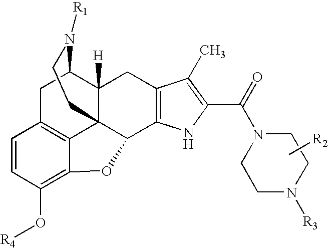

in which:

R₁ is hydrogen or alkyl;

R₂ is hydrogen or one or more alkyl groups;

R₃ is Rₜ or RₜX—; wherein Rₜ is hydrogen or optionally substituted alkyl, aryl, aryl-alkyl, cycloalkyl or heterocyclyl, and X is a linking group; and

R₄ is hydrogen or alkyl;

with the proviso that when R₄ is methyl and R₃ is methyl or hydroxyethyl then R₂ is not hydrogen.

Alkyl groups, including alkyl groups that are part of, for example, alkoxy or acyl groups, typically contain 1 to 6 carbon atoms, and may be linear or branched, such as methyl, ethyl, i-propyl, t-butyl, or i-pentyl. Aryl groups are typically phenyl, but may include bicyclic groups such as naphthyl. Aryl-alkyl groups include benzyl and phenylethyl. Cycloalkyl groups typically contain from 3 to 7 carbon atoms and include cyclobutyl and cyclohexyl. Heterocyclic groups may be monocyclic 5 to 7 membered rings containing up to three hetero atoms, such as pyridyl, pyrazinyl, pyrimidinyl, furyl, or imidazolyl, especially pyridyl, pyrimidyl; or bicyclic, especially heterocyclic rings fused to benzene rings, such as benzoxazolyl or benzimidazolyl. Aryl, cycloalkyl and heterocyclic groups may be optionally substituted by up to three substituents, which may suitably be selected from aryl, alkyl, alkoxy, halogen, hydroxy, oxo and cyano, or by linked substituents such as dioxymethylene.

R₁ and R₄ are typically hydrogen or alkyl, especially methyl.

Typical R₂ groups include hydrogen and alkyl, especially methyl, which may be present as dimethyl.

A particular group of compounds are those wherein when R₃ is methyl or hydroxyethyl then R₂ is not hydrogen.

R₃ groups are preferably Rₜ, RₜCO—, RₜNHCO—, or RₜSO₂—.

When Rₜ is an alkyl group suitable alkyl groups include methyl, ethyl, i-propyl, i-butyl, t-butyl, n-butyl, and i-pentyl, especially i-propyl. Alkyl groups may be substituted, for example by hydroxy, suitably as hydroxyethyl.

When Rₜ is an aryl or aryl-alkyl group, suitable aryl and aryl-alkyl groups include in particular phenyl, benzyl and phenylethyl.

When Rₜ is a cycloalkyl group, suitable cycloalkyl groups include cyclobutyl and cyclohexyl, especially cyclohexyl, and suitable heterocyclyl groups include furyl, pyridyl, pyrimidyl, benzoxazolyl, benzimidazolyl, and imidazolyl, especially pyridyl, pyrimidyl, benzoxazolyl, benzimidazolyl, and imidazolyl, more especially pyridyl and pyrimidyl.

Suitable values for X include —CO—, —OCO—, —NHCO—, —SO₂—, —CONH—, and —OCONH—, particularly —CO—, —NHCO—, and —SO₂—.

When R₃ is a group RₜX— suitable groups include ethyl-OOC—, t-butyl-O—CO—, i-butyl-CO—, n-butyl-CO—, i-pentyl-CO—, phenyl-CO—, benzyl-CO—, phenylethyl-CO—, cyclohexyl-CO—, methyl-NH—CO—, ethyl-NH—CO—, methyl-SO₂— and formyl. Especially suitable are phenyl-CO—, i-butyl-CO—, i-pentyl-CO—, benzyl-CO—, cyclohexyl-CO—, ethyl-NH—CO—, methanesulphonyl and formyl. Other suitable groups are ethyl-CO—, furyl-CO— and cyclobutyl-CO—.

Suitable optional substituents for cyclic R₃ groups include oxo, dioxymethylene, bromo, chloro, fluoro, hydroxy, cyano, methyl, methoxy, t-butyl and phenyl, especially dioxymethylene, chloro, fluoro, hydroxy, cyano, methyl, methoxy and t-butyl. A further suitable optional substituent is trifluoromethyl.

In a particular aspect R₃ is selected from hydrogen, methyl, ethyl, i-propyl, i-butyl, t-butyl, n-butyl, i-pentyl, hydroxyethyl, phenyl, benzyl, phenylethyl, cyclohexyl, cyclobutyl, pyridyl, pyrimidyl, benzoxazolyl, benzimidazolyl, imidazolyl, ethyl-CO—, ethyl-OOC—, t-butyl-O—CO—, i-butyl-CO—, n-butyl-CO—, i-pentyl-CO—, phenyl-CO—, furyl-CO—, benzyl-CO—, phenylethyl-CO—, cyclohexyl-CO—, cyclobutyl-CO—, methyl-NH—CO, ethyl-NH—CO—, methyl-SO₂— and formyl, where the cyclic R₃ groups are optionally substituted by one or more of oxo, dioxymethylene, bromo, chloro, fluoro, hydroxy, cyano, methyl, methoxy, t-butyl, trifluoromethyl and phenyl.

In another aspect R₃ is selected from hydrogen, methyl, ethyl, i-propyl, i-butyl, t-butyl, n-butyl, i-pentyl, hydroxyethyl, phenyl, benzyl, phenylethyl, cyclohexyl, pyridyl, pyrimidyl, benzoxazolyl, benzimidazolyl, imidazolyl, ethyl-OOC—, t-butyl-O—CO—, i-butyl-CO—, n-butyl-CO—, i-pentyl-CO—, phenyl-CO—, benzyl-CO—, phenylethyl-CO—, cyclohexyl-CO—, methyl-NH—CO, ethyl-NH—CO—, methyl-SO₂— and formyl, where the cyclic R₃ groups are optionally substituted by one or more of oxo, dioxymethylene, bromo, chloro, fluoro, hydroxy, cyano, methyl, methoxy, t-butyl, and phenyl.

In a further aspect R₃ is selected from hydrogen or optionally substituted alkyl, aryl, aryl-alkyl, cycloalkyl or heterocyclyl; or hydrogen or an optionally substituted alkyl, aryl, aryl-alkyl, cycloalkyl or heterocyclic group linked to N via a >C═O, —N—CO— or >SO₂ group.

The compounds of formula (I), or their salts or solvates, are preferably in pharmaceutically acceptable or substantially pure form. By pharmaceutically acceptable form is meant, inter alia, of a pharmaceutically acceptable level of purity excluding normal pharmaceutical additives such as diluents and carriers, and including no material considered toxic at normal dosage levels.

A substantially pure form will generally contain at least 50% (excluding normal pharmaceutical additives), preferably 75%, more preferably 90% and still more preferably 95% of the compound of formula (I) or its salt or solvate.

One preferred pharmaceutically acceptable form is the crystalline form, including such form in a pharmaceutical composition. In the case of salts and solvates the additional ionic and solvent moieties must also be non-toxic.

Examples of pharmaceutically acceptable salts of a compound of formula (I) include the acid addition salts with the conventional pharmaceutical acids, for example, maleic, hydrochloric, hydrobromic, phosphoric, acetic, fumaric, salicylic, citric, lactic, mandelic, tartaric, succinic, benzoic, ascorbic and methanesulphonic.

Salts or solvates of the compounds of formula (I) which are not pharmaceutically acceptable may be useful as intermediates in the production of pharmaceutically acceptable salts or solvates. Accordingly such salts or solvates also form part of this invention.

The compounds of formula (I) may exists in more than one stereoisomeric form, and the invention extends to all such forms as well as to their mixtures thereof, including racemates.

The compounds of formula (I), or salts or solvates thereof, may be prepared by the methods illustrated in the following general reaction schemes, or by modification thereof, using readily available starting materials, reagents and conventional synthetic procedures. These methods constitute a further aspect of the invention. If a particular enantiomer of a compound of the present invention is desired, it may be synthesised starting from the desired enantiomer of the starting material and performing reactions not involving racemization processes or it may be prepared by chiral synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxy, diastereomeric salts are formed with an appropriate optically active acid or base, followed by resolution of diastereomeric salts by fractional crystallization and subsequent recovery of the pure enantiomers.

The compounds of formula (I) and pharmaceutically acceptable derivatives thereof may be prepared by the processes described hereinafter, said processes constituting a further aspect of the invention. In the following description, the groups R₁, R₂, R₃ and R₄ are as defined for compounds of formula (I) unless otherwise stated.

Compounds of formula (I) may be prepared by reaction of acyl compounds of formula (II) with piperazines of formula (III) using the following reaction Scheme 1:

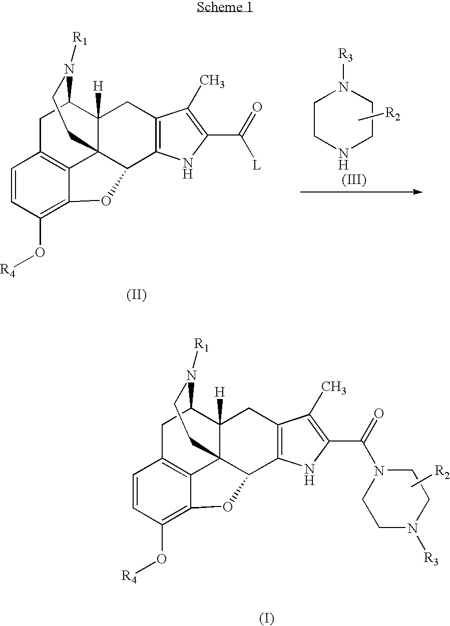

wherein R₁, R₂, R₃ and R₄ are as hereinbefore defined for compounds of formula (I) and L is a leaving group, for example halo, especially chloro; followed, if so desired, by conversion to a salt and/or solvate thereof.

The reaction of a compound of formula (II) with a compound of formula (III) is suitably carried out in the presence of a base, for example an organic base, such as an amine, for example triethylamine, diisopropylethylamine, pyridine or dicyclohexylamine. A particular amine that may be mentioned is triethylamine. The reaction is suitably carried out in the presence of a solvent, for example an aprotic solvent such as N,N-dimethylformamide, tetrahydrofuran, N-methylpyrrolidinone, or dimethoxyethane; or a chlorinated solvent such as dichloroethane or dichloromethane. A particular solvent that may be mentioned is tetrahydrofuran. Suitable reaction temperatures include 10-40° C., more particularly 15-25° C.

Compounds of formula (II) may be prepared according to processes known in the art for the preparation of acyl groups, for example S. Patai, The Chemistry of Acyl Halides, pp 35-78, Interscience, New York, 1972. The compounds of formula (II) when L is chloro may be obtained for example by reacting a ketone of formula (IV) with a hydrazone of formula (V), in the presence of Zn and CH₃COONa in CH₃COOH as solvent (Khimiya Geterot. Soed., 1972, 342), followed by treatment with sodium hydroxide to give the sodium carboxylate. Conversion to the corresponding acyl chloride is achieved by treatment with oxalyl chloride, as shown in scheme 2:

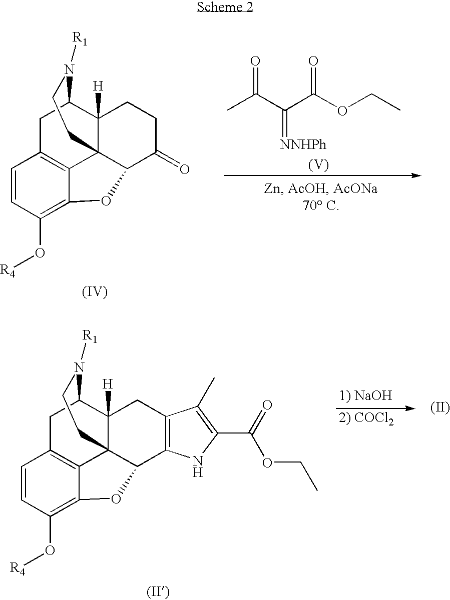

Suitable starting materials of formula (IV) include the morphinan hydrocodone (R₁ and R₄ are methyl), which is commercially available, and other compounds which are known in the literature. The other substituents specified for R₁ and R₄ in formula (I) may incorporated and/or converted by conventional substitution reactions before or after the coupling of compounds (II) and (III). For example, a compound of formula (I) or formula (II) in which R₁ is alkyl may be converted into another compound of formula (I) or formula (II) respectively in which R₁ is hydrogen by conventional methods. Similarly a compound of formula (I) or formula (II) in which R₁ is hydrogen may be converted into a compound of formula (I) or formula (II) in which R₁ is alkyl by conventional substitution reactions. Similarly compounds of formula (I) or formula (II) in which R₄ is alkyl may be converted to the corresponding compounds in which R₄ is hydrogen, and compounds in which R₄ is hydrogen may be converted to corresponding compounds in which R₄ is alkyl.

The compounds of formula (I) may be alternatively obtained starting from ketones of formula (IV) and hydrazones of formula (VI) prepared using as starting materialcompounds prepared by reacting ethylacetoacetate with the appropriate substituted piperazines of general formula (III). The resulting acetoacetamides of general formula (VII) are in turn reacted with phenyldiazonium salt to obtain the corresponding hydrazones of general formula (VI) as shown in Scheme 3:

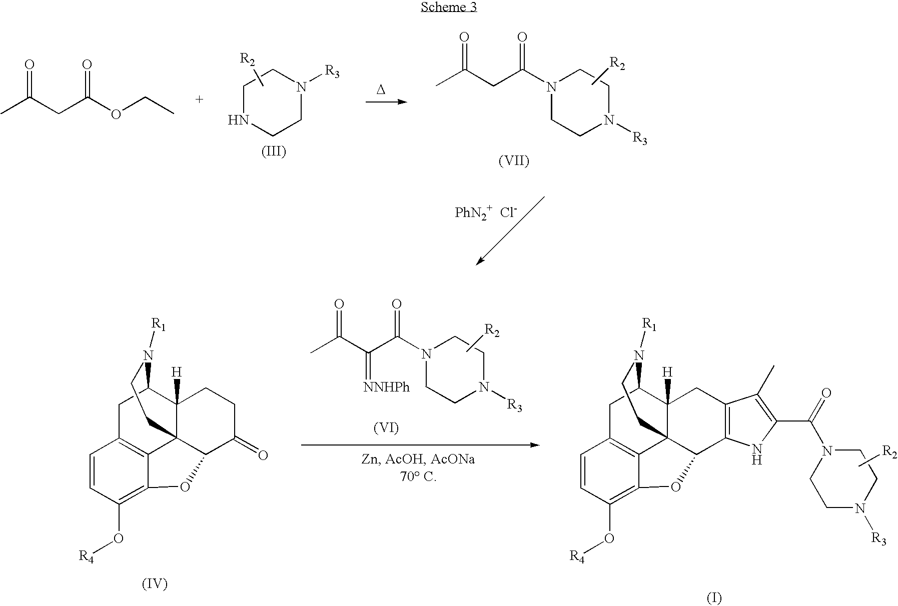

The substituted piperazines of formula (V) are either commercially available or readily synthesised by conventional methods from commercially available materials.

Alternatively, compounds of general formula (I) can be also obtained from compounds of general formula (I′), in which R₁ is alkyl and R₃ is hydrogen, by alkylation or acylation of the piperazine basic nitrogen using conventional methods. Compounds of general formula (I′) can be in turn obtained from compounds of general formula (I″), synthesised as described above for compounds of general formula (I), where R₃″ is a suitable protecting group, for example, 9-fluorenylmethoxycarbonyl (FMOC) or N-t-butoxycarbonyl (BOC), that can be easily removed by conventional methods to give the desired compounds of general formula (I′) as shown in Scheme 4.

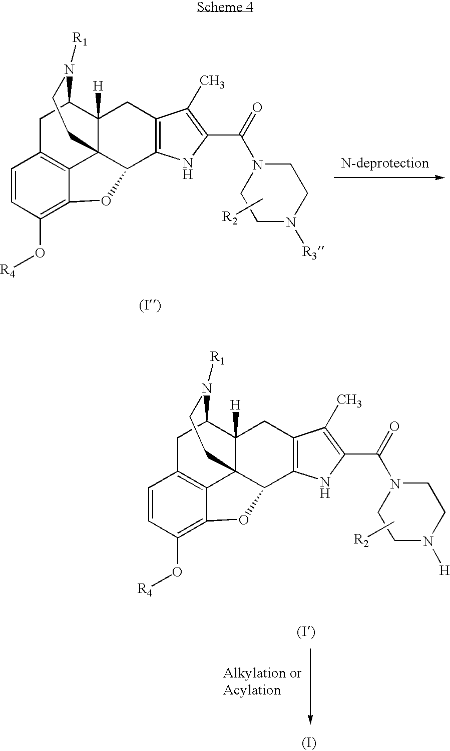

In a further aspect the present invention provides for novel intermediates of formulae (II), (II′), (III), (V), (VI), (VII), (I′) and (I″).

The compounds of formula (I) may be converted into their pharmaceutically acceptable salts by reaction with the appropriate organic or mineral acids.

Solvates, including hydrates, of the compounds of formula (I) may be formed by crystallization or recrystallization from the appropriate solvent. For example, hydrates may be formed by crystallization or recrystallization from aqueous solutions, or solutions in organic solvents containing water.

In general compounds of formula (I) acting as selective delta receptor ligands may be useful as analgesics and antihyperalgesics for different pain conditions, immunosuppressants to prevent rejection in organ transplant and skin graft, anti-allergic and anti-inflammatory agents, brain cell protectant, for the treatment of drug and alcohol abuse, to decrease gastric secretion, for the treatment of diarrhoea, cardiovascular and respiratory diseases, cough and respiratory depression, mental illness, epileptic seizures and other neurologic disorders (herein after referred to as ‘Conditions’). In particular, the activity of the compounds of formula (I) as delta agonists in standard tests indicates that they are of potential therapeutic utility as analgesic agents for the amelioration or elimination of pain.

Accordingly the present invention provides a method for the treatment and/or prophylaxis of one or more of the Conditions in mammals, particularly humans, which comprises administering to the mammal in need of such treatment and/or prophylaxis an effective amount of a compound of formula (I) or a pharmaceutically acceptable salt or solvate thereof.

The present invention also provides a compound of formula (I), or a pharmaceutically acceptable salt or solvate thereof, for use as an active therapeutic substance. In particular the present invention also provides a compound of formula (I) or a pharmaceutically acceptable salt thereof for use in the treatment and/or propylaxis of one or more of the Conditions.

The present invention further provides a pharmaceutical composition comprising a compound of formula (I), or a pharmaceutically acceptable salt or solvate thereof, and a pharmaceutically acceptable carrier.

The present invention also provides the use of a compound of formula (I), or a pharmaceutically acceptable salt or solvate thereof, in the manufacture of a medicament for the treatment and/or prophylaxis of one or more of the Conditions.

Such a medicament, and a composition of this invention, may be prepared by admixture of a compound of the invention with an appropriate carrier. It may contain a diluent, binder, filler, disintegrant, flavouring agent, colouring agent, lubricant or preservative in conventional manner.

These conventional excipients may be employed for example as in the preparation of compositions of known agents for treating the Conditions.

Preferably, a pharmaceutical composition of the invention is in unit dosage form and in a form adapted for use in the medical or veterinarial fields. For example, such preparations may be in a pack form accompanied by written or printed instructions for use as an agent in the treatment of the Conditions.

The suitable dosage range for the compounds of the invention depends on the compound to be employed and on the condition of the patient. It will also depend, inter alia, upon the relation of potency to absorbability and the frequency and route of administration.

The compound or composition of the invention may be formulated for administration by any route, and is preferably in unit dosage form or in a form that a human patient may administer to himself in a single dosage. Advantageously, the composition is suitable for oral, rectal, topical, parenteral, intravenous or intramuscular administration. Preparations may be designed to give slow release of the active ingredient.

Compositions may, for example, be in the form of tablets, capsules, sachets, vials, powders, granules, lozenges, reconstitutable powders, or liquid preparations, for example solutions or suspensions, or suppositories.

The compositions, for example those suitable for oral administration, may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable setting agents such as sodium lauryl sulphate.

Solid compositions may be obtained by conventional methods of blending, filling, tabletting or the like. Repeated blending operations may be used to distribute the active agent throughout those compositions employing large quantities of fillers. When the composition is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The composition may also be in the form of an ingestible capsule, for example of gelatin containing the compound, if desired with a carrier or other excipients.

Compositions for oral administration as liquids may be in the form of, for example, emulsions, syrups, or elixirs, or may be presented as a dry product for reconstitution with water or other suitable vehicle before use. Such liquid compositions may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; aqueous or non-aqueous vehicles, which include edible oils, for example almond oil, fractionated coconut oil, oily esters, for example esters of glycerine, or propylene glycol, or ethyl alcohol, glycerine, water or normal saline; preservatives, for example methyl or propyl p-hydroxybenzoate or sorbic acid; and if desired conventional flavouring or colouring agents.

The compounds of this invention may also be administered by a non-oral route. In accordance with routine pharmaceutical procedure, the compositions may be formulated, for example for rectal administration as a suppository. They may also be formulated for presentation in an injectable form in an aqueous or non-aqueous solution, suspension or emulsion in a pharmaceutically acceptable liquid, e.g. sterile pyrogen-free water or a parenterally acceptable oil or a mixture of liquids. The liquid may contain bacteriostatic agents, anti-oxidants or other preservatives, buffers or solutes to render the solution isotonic with the blood, thickening agents, suspending agents or other pharmaceutically acceptable additives. Such forms will be presented in unit dose form such as ampoules or disposable injection devices or in multi-dose forms such as a bottle from which the appropriate dose may be withdrawn or a solid form or concentrate which can be used to prepare an injectable formulation.

The compounds of this invention may also be administered by inhalation, via the nasal or oral routes. Such administration can be carried out with a spray formulation comprising a compound of the invention and a suitable carrier, optionally suspended in, for example, a hydrocarbon propellant.

Preferred spray formulations comprise micronised compound particles in combination with a surfactant, solvent or a dispersing agent to prevent the sedimentation of suspended particles. Preferably, the compound particle size is from about 2 to 10 microns.

A further mode of administration of the compounds of the invention comprises. transdermal delivery utilising a skin-patch formulation. A preferred formulation comprises a compound of the invention dispersed in a pressure sensitive adhesive which adheres to the skin, thereby permitting the compound to diffuse from the adhesive through the skin for delivery to the patient. For a constant rate of percutaneous absorption, pressure sensitive adhesives known in the art such as natural rubber or silicone can be used.

As mentioned above, the effective dose of compound depends on the particular compound employed, the condition of the patient and on the frequency and route of administration. A unit dose will generally contain from 20 to 1000 mg and preferably will contain from 30 to 500 mg, in particular 50, 100, 150, 200, 250,300, 350, 400, 450, or 500 mg. The composition may be administered once or more times a day for example 2, 3 or 4 times daily, and the total daily dose for a 70 kg adult will normally be in the range 100 to 3000 mg. Alternatively the unit dose will contain from 2 to 20 mg of active ingredient and be administered in multiples, if desired, to give the preceding daily dose.

No unacceptable toxicological effects are expected with compounds of the invention when administered in accordance with the invention.

The activity of the compounds of the present invention as selective delta ligands is determined in radioligand binding assays as described below.

### Pharmacological Data

The activity of the compounds of the present invention as selective delta ligands is determined in radioligand binding assays using cloned human delta, mu and kappa opioid receptors stably expressed in cell lines as described below.

CHO cells were subjected to stable transfection with cDNA encoding the human delta and mu opioid receptors. Clones were grown in suspension culture in serum free media. Selection was performed by growth in the absence of nucleotides.

Human kappa opioid receptors were stably expressed in HEK cells. Cells were grown in adhesion in E-MEM supplemented with 10% FBS and 2 mM L-glutamine, G418 was included for selection.

Membranes were prepared as previously described (J. Med. Chem. 1997, 40 3192). The binding of the preferential delta ligand [³H]-[D-Ala²,D-Leu⁵]-enkephalin (DADLE) was evaluated at its K_{D} concentration (0.7 nM). The binding of the mu ligand [³H]-DAMGO (Eur. J. Pharmacol., 1989, 166, 213) and of the kappa ligand [³H]-U69593 (Excerpta Medica, 1990, 211) were carried out at 0.5 nM. Non specific binding was determined in the presence of 10 uM of naloxone. Binding data were expressed as percentage of inhibition and fitted the following equation: f(x)=100·X/(IC₅₀+X) where X is the cold drug concentration value. The IC₅₀ values obtained were used to calculate the inhibitory constants (Kᵢ) according to the Cheng and Prusoff relation (Biochem. Pharmacol., 1973, 22, 3099).

The most potent compounds described in the present invention showed affinities for the delta receptor ranging from 0.3 to 10 nM with delta selectivity ranging from 15 to 400 times in respect to the other opioid receptor types.

For example, the compounds of Examples 6 and 23 show a Ki delta=1.0 nM (Ki mu/Ki delta=437) and Ki delta=0.6 (Ki mu/Ki delta=1010), respectively.

The following Procedures X and Y and Example 15 illustrate the preparation of compounds of general formula (I) of the present invention. The compounds of the Examples 1 to 14 and 16 to 39 and 48 to 52 are obtained using the general procedure as described in Example 15 (Method A), starting from the corresponding acyl chloride of general formula (II) and the corresponding known substituted piperazines of general formula (III). Procedures X and Y illustrate the preparation of intermediates of formulae (II) and (V).

The compounds of the remaining Examples 41 to 47 are obtained using the same procedure as described in Procedures P and Q and Example 40 (Method B), starting from the corresponding acyl chloride and the piperazines of general formula (I′) by removal of a protecting group from compounds of general formula (I″,) synthesised as described above using Method A.

The compounds obtained in the Examples 1 to 52 are summarised in Table 1.

### Procedure X

### Preparation of sodium salt of [8R-(4bS*,8α,8a β,12b β)]-7,10-dimethyl-1-methoxy-1-5,6,7,8,12,12b-hexahydro-(9H)-4,8-methanobenzofuro[3,2-e]-pyrrolo[2,3-g]isoquinoline-11-carboxylic acid (compound of Formula II′ wherein R₁=Me, and R₄=Me).

To a solution of hydrocodeinone (25 g, MW 335.8, 74 mmol) in 400 ml of AcOH, AcONa (20 g, MW 82, 224 mmol) and 3-oxo-2-(phenyl-hydrazono)-butyric acid ethyl ester (V, see below) (34.6 g, MW 234.25, 147 mmol) were added. The mixture was heated to 50-60° C. and then Zn (16 g, MW 65.4, 244 mmol) was added portionwise, maintaining the temperature below 90° C. The mixture was then heated to 100° C. for 8 hours. The slurry was poured in a mixture containing 30% NH₄OH (200 ml) and 400 g of brine and then extracted with AcOEt (3×200 ml). The organic layer was dried over Na₂SO₄ and the solvent was removed under vacuum. The residue was purified by chromatography on silica gel eluting with AcOEt/MeOH/NH₄OH 90/10/1 to afford the [8R-(4bS*,8 α,8a β,12b β]-7,10-dimethyl-1-methoxy-11-ethyloxycarbonyl-5,6,7,8,12,12b-hexahydro-(9H)-4,8-methanobenzofuro[3,2-e]-pyrrolo[2,3-g]isoquinoline (12 g, PM 408.5, 30 mmol).

A mixture of this compound (5 g, 12 mmol) and 1M NaOH (30 ml) in 60 ml of EtOH 96% was stirred at 80° C. for 5 hours. The solvent was evaporated under vacuum and the residue was triturated in water (30 ml). After filtration, the solid was dried under vacuum at 50° C. for one night affording the title compound (4 g, MW 402.4, 10 mmol).

Treatment with oxalyl chloride (see Method A, below) provides the compound of Formula II wherein R₁=Me, R₄=Me, and L=Cl.

Procedure Y

3-Oxo-2-(phenyl-hydrazono)-butyric acid ethyl ester (Compound V)

In a flask containing 250 ml of 5 N HCl at the temperature of 0-5° C., 46.5 g of aniline (0.5 moles) were added under nitrogen flow. Then a solution of 36.5 g of NaNO₂ (0.53 moles) in 125 ml of water was added dropwise maintaining the temperature at 5-10° C. At the end of the addition the solution was adjusted to pH 4-5 with solid sodium acetate. The obtained solution was then transferred in a dropping funnel and added dropwise to a solution of ethylacetoacetate (65 g, 0.5 moles) in 375 ml of EtOH (95°), 100 ml of water and sodium acetate (0.73 moles) maintaining the temperature below 5-10° C. Then the reaction was warmed up to room temperature, and it was further stirred for two hours. 500 ml of water were added and after 30 minutes stirring, the suspension was filtered under vacuum. The orange-yellow solid was dried under vacuum at room temperature overnight to obtain 68 g of the title compound.

### Example 15 (Method A)

### [8R-(4bS*,8 α,8a β,12b β)]-11-(4-(4-Chlorophenyl)piperazin-1-yl)carbonyl-1-methoxy-7,10-dimethyl-5,6,7,8,12,12b-hexahydro-(9H)-4,8-methanobenzofuro[3,2-e]-pyrrolo[2,3-g]isoquinoline

Oxalyl chloride (4.0 g, 31 mmol) was added dropwise at 0° C. to a suspension of sodium salt prepared as described above in Procedure X (1.7 g, MW 402, 4.23 mmol) in THF (300 ml). The mixture was stirred 4 hours at room temperature (or to complete solution) and then was concentrated under vacuum. The residue was dissolved in THF (100 ml) and the solution of 4-chlorophenylpiperazine hydrochloride (1.25 g, MW 269.6, 4.65 mmol) in THF (5 ml) and TEA (2.6 ml) were added dropwise. The mixture was stirred at room temperature for 4 hours, then the solvent was removed in vacuo. The residue was dissolved in AcOEt (50 ml×2) and washed with 1N NaOH (50 ml). The organic layer was dried over Na₂SO₄ and evaporated under vacuum. The product was purified by chromatography on silica gel eluting with AcOEt/MeOH/NH₄OH 90/10/1 to afford mg 0.8 g of the title compound.

### Procedure P

### [8R-(4b S, 8 α, 8a β 12b β)]-11-(4-Tertbutoxycarbonylpiperazin-1-yl)carbonyl-1-methoxy-7,10-dimethyl-5,6,7,8,12,12b-hexahydro-(9H)4,8-methanobenzofuro[3,2-e]pyrrolo[2,3-g]isoquinoline (Compound of Formula I″, R₁=Me, R₄=Me, R₂=H, R₃=tert-butoxycarbonyl)

Oxalyl chloride (1.1 ml, 12 mmol) was added dropwise at 0° C. to a suspension of sodium salt prepared as described above in Procedure X (0.5 g, MW 402, 1.2 mmol) in THF (30 ml). The mixture was stirred 4 hours at room temperature and then was concentrated under vacuum.

The residue was dissolved in THF (10 ml) then N-BOC-piperazine (II) (mg 250, MW 185, 1.3 mmol) in THF (2 ml) and TEA (500 μl, 3.6 mmol) were added. The mixture was stirred at room temperature for 4 hours, then the solvent was removed in vacuo. The residue was dissolved in AcOEt (20 ml×2) and washed with 1N NaOH (5 ml). The organic layer was dried over Na₂SO₄ and evaporated under vacuum. The product was purified by chromatography on silica gel eluting with AcOEt/MeOH/NH₄OH 90/10/1 to afford mg 500 of the title compound.

### Procedure Q

### [8R-(4b S, 8 α, 8a β, 12b β)]-11-(piperazin-1-yl)carbonyl-1-methoxy-7,10-dimethyl-5,6,7,8,12,12b-hexahydro-(9H)4,8-methanobenzofuro[3,2-e]pyrrolo[2,3-g]isoquinoline (Compound of Formula I′, R₁=Me, R₄=Me, R₂=H)

N-BOC derivative of Procedure P (500 mg, MW 520.63, 1 mmol) was dissolved in CH₂Cl₂ (8 ml) and then TFA (0.5 ml) was added slowly. The reaction was stirred overnight at room temperature, and then the solvent and the excess of TFA were evaporated under vacuo. The residue was dissolved in CH₂Cl₂ (10 ml×2) and washed with a saturated solution of Na₂CO₃ (10 ml). The organic layer was dried over Na₂SO₄ and evaporated under vacuum. The product was triturated with Et₂O to give the title compound (300 mg, MW 420, 0.71 mmol).

### Example 40 (Method B)

### [8R-(4b S, 8 α, 8a β, 12b β)]-11-[4-(2,4-Dimethoxybenzoyl)piperazin-1-yl]carbonyl-1-methoxy-7,10-dimethyl-5,6,7,8,12,12b-hexahydro-(9H)4,8-methanobenzofuro[3,2-e]pyrrolo[2,3-g]isoquinoline

To a solution of the piperazino derivative prepared in Procedure Q (100 mg, MW 420, 0.26 mmol) in THF (3 ml), TEA (37 μl, MW 101, 0.26 mmol) and 2,4-dimethoxybenzoyl chloride (52 mg, MW 200.62, 0.26 mmol) were added. The mixture was stirred at room temperature for 15 hours. 1N NaOH (2 ml ) was added and the mixture was extracted with AcOEt (6 ml×2). The organic layer was dried over Na₂SO₄ and evaporated under vacuum. The product was purified by chromatography on silica gel eluting with AcOEt/MeOH/NH₄OH 80/20/1 to afford the title compound (68 mg, MW 612, 0.11 mmol).

TABLE 1

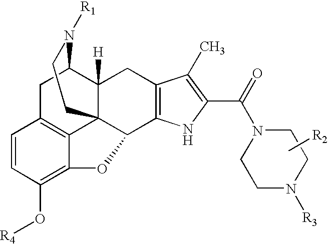

| | | | | | | mass |
|---|---|---|---|---|---|---|
| | | | | | | ESI POS; AQA; solvent: |
| | | | | | [a]_{D}^{20§}/ | methanol/spray 3 kV/ |
| Ex. | R₁ | R₂ | R₃ | R₄ | (MP ° C.) | skimmer: 20 V/probe 135 C. |
| 1 | Me | H | CH₂Ph | Me | −384.72 | * 538 (M+.); 447; |
| | | | | | (172.5-177.4) | 405; 379; 363; 175; |
| | | | | | | 146; 91 |
| 2 | Me | H | Ph | Me | | * 524 (M+.); 405; |
| | | | | | | 390; 379; 362; 161; |
| | | | | | | 145; 132 |
| 3 | Me | H | i-Pr | Me | −431.9 | * 490 (M+.); 405; |
| | | | | | (166.3-167.7) | 363; 98 |
| 5 | Me | H | 2-Py | Me | −461.4 | 526 (MH+.) |
| | | | | | (253-255) | |
| 5 | Me | H | 2,6-Me—Ph | Me | (165-168) | * 552 (M+.); 405; |
| | | | | | | 379; 362; 189; 173; |
| | | | | | | 160 |
| 6 | Me | H | 2,4-Me—Ph | Me | (173-175) | * 552 (M+.); 379; |
| | | | | | | 363; 190; 173; 160 |
| 7 | Me | H | 2-Me—Ph | Me | — | 539 (MH+) |
| 8 | Me | H | Cy | Me | (130-132) | 531 (MH+) |
| 9 | Me | H | 4-MeO—Ph | Me | −486.2 | * 554 (M+.); 363; |
| | | | | | | 192; 175; 162; 150 |
| 10 | Me | H | 3,4-(OCH₂O)— | Me | | * 582 (M+.), 447, |
| | | | PhCH₂ | | | 379; 363; 219; 190; |
| | | | | | | 176; 135 |
| 11 | Me | H | COPh | Me | −263.0 | 553 (MH+) |
| 12 | Me | H | 4-F—Ph | Me | | 543 (MH+) |
| 13 | Me | H | 2-MeO—Ph | Me | | 555 (MH+) |
| 14 | Me | H | 2-Cl—Ph | Me | — | 559 (MH+) |
| 15 | Me | H | 4-Cl—Ph | Me | −435.8 | 559 (MH+) |
| | | | | | (189-192) | |
| 16 | Me | H | 4-t-Bu—Ph | Me | — | 581 (MH+) |
| 17 | Me | H | 4-Cl-PhCH₂ | Me | — | 574 (MH+) |
| 18 | Me | H | PhCH₂CH₂ | Me | −421.7 | 553 (MH+) |
| | | | | | (259-260) | |
| 19 | Me | H | 4-F—PhCH₂ | Me | | 557 (MH+) |
| 20 | Me | H | 4-MeO-PhCH₂ | Me | −359.6 | 569 (MH+) |
| | | | | | (150-152) | |
| 21 | Me | H | 3-Me—PhCH₂ | Me | — | 553 (MH+) |
| 22 | Me | H | 4-Me—PhCH₂ | Me | — | 553 (MH+) |
| 23 | Me | H | 2,4-MeO—Ph | Me | −388.5 | 585 (MH+) |
| | | | | | (155-160) | |
| 24 | Me | 3-Me | Ph | Me | — | 539 (MH+) |
| 25 | Me | H | CHO | Me | −473.5 | 477 (MH+) |
| | | | | | (205-210) | |
| 26 | Me | H | 3-Cl—Ph | Me | — | 559 (MH+) |
| 27 | Me | H | 2-MeO-5-Cl—Ph | Me | — | 589 (MH+) |
| 28 | Me | 3,5- | H | Me | — | 477 (MH+) |
| | | Me | | | | |
| | | (cis) | | | | |
| 29 | Me | H | 3-Cl—Ph | Me | (165-167) | 559 (MH+) |
| 30 | Me | H | 4-OH—Ph | Me | — | 541 (MH+) |
| 31 | Me | H | 4-Cl—PhCO | Me | — | 588 (MH+) |
| 32 | Me | H | 4-MeO—PhCO | Me | — | 583 (MH+) |
| 33 | Me | H | MeSO₂ | Me | — | 527 (MH+) |
| 34 | Me | H | 4-CN—Ph | Me | — | 550 (MH+) |
| 35 | Me | H | MeCH₂NHCO | Me | — | 582 (MH+) |
| 36 | Me | H | 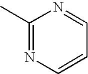 | Me | — | 527 (MH+) |
| 37 | Me | H | PhCH₂CO | Me | — | 567 (MH+) |
| 38 | Me | H | 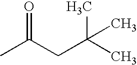 | Me | — | 547 (MH+) |
| 39 | Me | H | CH₃(CH₂)₃CO | Me | — | 533 (MH+) |
| 40 | Me | H | 2,4-MeO—PhCO | Me | −421.5 | 613 (MH+) |
| | | | | | (190-193) | |
| 41 | Me | H | i-BuCO | Me | — | 533 (MH+) |
| 42 | Me | H | PhCH₂CH₂CO | Me | — | 581 (MH+) |
| 43 | Me | H | CyCO | Me | — | 559 (MH+) |
| 44 | Me | H | CH₃CH₂CO | Me | 505 (MH+) | |
| 45 | Me | H | 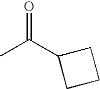 | Me | | 531 (MH+) |
| 46 | Me | H | 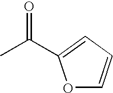 | Me | | 543 (MH+) |
| 47 | Me | H | 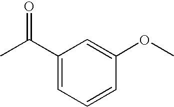 | Me | | 583 (MH+) |
| 48 | Me | H | 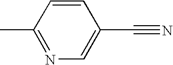 | Me | | 551 (MH+) |
| 49 | Me | H | 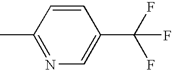 | Me | | 594 (MH+) |
| 50 | Me | H | 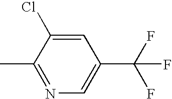 | Me | | 628 (MH+) |
| 51 | Me | H | 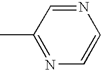 | Me | | 527 (MH+) |
| 52 | Me | H | 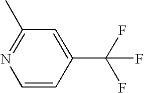 | Me | | 594 (MH+) |

^{§}c = 0.1%, solvent: MeOH;
* EI; TSQ 700; source 180 C;70 V;200 uA Table 3 illustrates the intermediates of formula (II) and (III) for the preparation of Examples 1-39 and 48-52 by Method A.

TABLE 3

| | |
|---|---|
| | (II) |
| 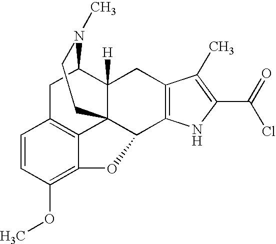 | |

| | |
|---|---|
| | (III) |
| 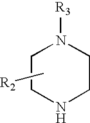 | |

| | Compound (III) | |
|---|---|---|
| Ex | R₂ | R₃ |
| 1 | H | CH₂Ph |
| 2 | H | Ph |
| 3 | H | i-Pr |
| 4 | H | 2-Py |
| 5 | H | 2,6-Me—Ph |
| 6 | H | 2,4-Me—Ph |
| 7 | H | 2-Me—Ph |
| 8 | H | Cy |
| 9 | H | 4-MeO—Ph |
| 10 | H | 3,4-(OCH₂O)—PhCH₂ |
| 11 | H | COPh |
| 12 | H | 4-F—Ph |
| 13 | H | 2-MeO—Ph |
| 14 | H | 2-Cl—Ph |
| 15 | H | 4-Cl—Ph |
| 16 | H | 4-t-Bu—Ph |
| 17 | H | 4-Cl—PhCH₂ |
| 18 | H | PhCH₂CH₂ |
| 19 | H | 4-F—PhCH₂ |
| 20 | H | 4-MeO—PhCH₂ |
| 21 | H | 3-Me—PhCH₂ |
| 22 | H | 4-Me—PhCH₂ |
| 23 | H | 2,4-MeO—Ph |
| 24 | 3-Me | Ph |
| 25 | H | CHO |
| 26 | H | 3-Cl—Ph |
| 27 | H | 2-MeO-5-Cl—Ph |
| 28 | 3,5-Me (cis) | H |
| 29 | H | 3-Cl—Ph |
| 30 | H | 4-OH—Ph |
| 31 | H | 4-Cl—PhCO |
| 32 | H | 4-MeO—PhCO |
| 33 | H | MeSO₂ |
| 34 | H | 4-CN—Ph |
| 35 | H | MeCH₂NHCO |
| 36 | H | 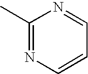 |
| 37 | H | PhCH₂CO |
| 38 | H | 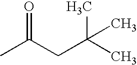 |
| 39 | H | CH₃(CH₂)₃CO |
| 48 | H | 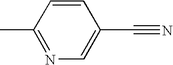 |
| 49 | H | 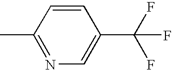 |
| 50 | H | 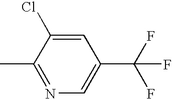 |
| 51 | H | 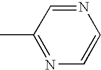 |
| 52 | H | 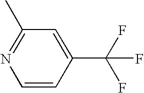 |

Examples 40-47 are prepared according to Method B by reacting the compound of formula (I′) wherein R₁=Me, R₄=Me and R₂=H (prepared according to Procedure Q):

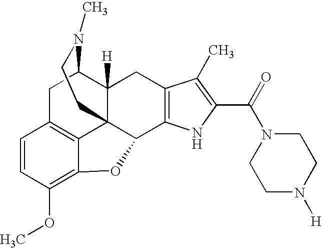

with the appropriate acylating reagent as indicated in Table 4.

TABLE 4

| Ex | Acylating reagent |
|---|---|
| 40 | 2,4-MeO—PhCOCl |
| 41 | i-BuCO |
| 42 | PhCH₂CH₂COCl |
| 43 | CyCOCl |
| 44 | CH₃CH₂COCl |
| 45 | 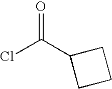 |
| 46 | 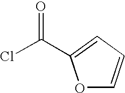 |
| 47 | 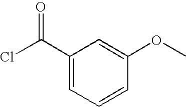 |

1. A compound, or a solvate or salt thereof, of formula (I): 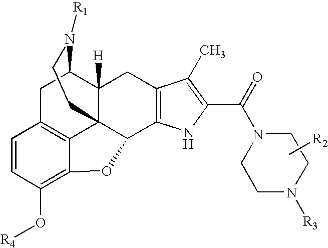
in which:
R₁ is hydrogen or alkyl;
R₂ is hydrogen or one or more alkyl groups;
R₃ is Rₜ or RₜX—; wherein Rₜ is hydrogen or optionally substituted alkyl, aryl, aryl-alkyl, cycloalkyl or heterocyclyl and X is a linking group; and
R₄ is hydrogen or alkyl;
with the proviso that when R₄ is methyl and R₃ is methyl or hydroxyethyl, then R₂ is not hydrogen.

2. A compound according to claim 1 in which Rₜ is hydrogen; optionally substituted methyl, ethyl, i-propyl, i-butyl, t-butyl, n-butyl, or i-pentyl; optionally substituted phenyl, benzyl or phenylethyl; optionally substituted cyclobutyl or cyclohexyl; or optionally substituted furyl, pyridyl, pyrimidyl, benzoxazolyl, benzimidazolyl or imidazolyl.

3. A compound according to claim 1 in which R₃ is selected from hydrogen, methyl, ethyl, i-propyl, i-butyl, t-butyl, n-butyl, i-pentyl, hydroxyethyl, phenyl, benzyl, phenylethyl, cyclohexyl, cyclobutyl, pyridyl, pyrimidyl, benzoxazolyl, benzimidazolyl, imidazolyl, ethyl-CO—, ethyl-OOC—, t-butyl-O—CO—, i-butyl-CO—, n-butyl-CO—, i-pentyl-CO—, phenyl-CO—, furyl-CO—, benzyl-CO—, phenylethyl-CO—, cyclohexyl-CO—, cyclobutyl-CO—, methyl-NH—CO, ethyl-NH—CO—, methyl-SO₂— and formyl, where the cyclic R₃ groups are optionally substituted by one or more of oxo, dioxymethylene, bromo, chloro, fluoro, hydroxy, cyano, methyl, methoxy, t-butyl, trifluoromethyl and phenyl.

4. A compound according to any one of claims claim 1 to 3 in which R₂ is selected from hydrogen, methyl and dimethyl.

5. A compound according to any one of claims 1 to 4 in which R₁ and R₄ are independently selected from hydrogen and methyl.

6. A compound according to claim 1 which is any one of compounds 1 to 52 in the Chemical Table herein.

7. A method for the treatment and/or prophylaxis of one or more of the Conditions in mammals, particularly humans, which comprises administering to the mammal in need of such treatment and/or prophylaxis an effective amount of a compound according to any one of claims 1 to 5 or a pharmaceutically acceptable salt or solvate thereof.

8. A compound according to any one of claims 1 to 5, or a pharmaceutically acceptable salt or solvate thereof, as an active therapeutic substance.

9. Use of a compound according to any one of claims 1 to 5, or a pharmaceutically acceptable salt or solvate thereof, in the manufacture of a medicament for the treatment and/or prophylaxis of one or more of the Conditions.

10. A process for the preparation of a compound of formula (I) according to claim 1 which process comprises:
reacting a compound of formula (II): 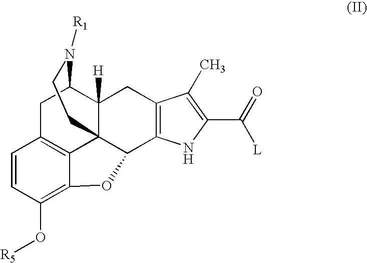
with a compound of formula (III): 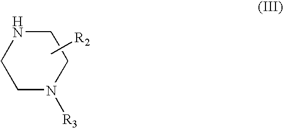
and, if desired, converting to a salt and/or solvate thereof;
wherein L is a leaving group and R₁, R₂, R₃ and R₄, are as defined in claim 1.
